(19) United States
(12) Patent Application Publication
(10) Pub. No.: US 2025/0127135 A1
(43) Pub. Date: Apr. 24, 2025

(54) GRAPEVINE PLANT NAMED ‘JB 12-24-E4-12’

(71) Applicant: Early Morning LLC, Greendale, IN (US)

(72) Inventor: Patterson Jeffrey BLOODWORTH, Hillsborough, NC (US)

(73) Assignee: Early Morning LLC, Greendale, IN (US)

(21) Appl. No.: 18/489,982

(22) Filed: Oct. 19, 2023

(51) Int. Cl. A01H 6/88; A01H 5/08

(52) U.S. Cl. CPC A01H 6/88; A01H 5/08

(57) ABSTRACT

A new grapevine plant particularly distinguished by large, seedless, attractively colored berries borne in well-filled clusters, unlike traditional seeded muscadines, which are marketed as individual berries, typical muscadine aroma and flavor combined with relatively thin, more edible skin and juicier flesh compared to most traditional seeded muscadine cultivars, is disclosed.

### BACKGROUND OF THE NEW PLANT

The present invention relates to a new and distinct variety of grapevine hereinafter referred to by the variety name ‘JB 12-24-E4-12’.

‘JB 12-24-E4-12’ arose from a controlled cross in 2012 in Hillsborough, North Carolina between a proprietary and unpatented grapevine named ‘JB 99-1-4-15’ (the results of a cross between Supreme and an Open Pollination) female parent, and a proprietary and unpatented grapevine named ‘JB 06-23-1-4’ (Ga 9-11-2 x JB 03-20-1-21). Seeds and seedlings from the cross were sown and planted in Wasco, California in 2013 and ‘JB 12-24-E4-12’ was selected in September 2015.

‘JB 12-24-E4-12’ was first asexually propagated in 2015 in Wasco, California by softwood cuttings. ‘JB 12-24-E4-12’ was found to reproduce true to type in successive generations of asexual propagation via softwood cuttings.

### SUMMARY

The following are the most outstanding and distinguishing characteristics of this new variety when grown under normal horticultural practices in Hillsborough, North Carolina.
1. Large, seedless, attractively colored berries borne in well-filled clusters, unlike traditional seeded muscadines, which are marketed as individual berries.
2. Typical muscadine aroma and flavor combined with relatively thin, more edible skin and juicier flesh compared to most traditional seeded muscadine cultivars.

### DESCRIPTION OF THE PHOTOGRAPHS

This new grapevine plant is illustrated by the accompanying photographs which shows the plant's overall plant habit. The colors shown are as true as can be reasonably obtained by conventional photographic procedures.

FIG. 1 shows the overall plant, leaves and fruit of ‘JB 12-24-E4-12’ at five years old, taken in Hillsborough, North Carolina in September of 2021.

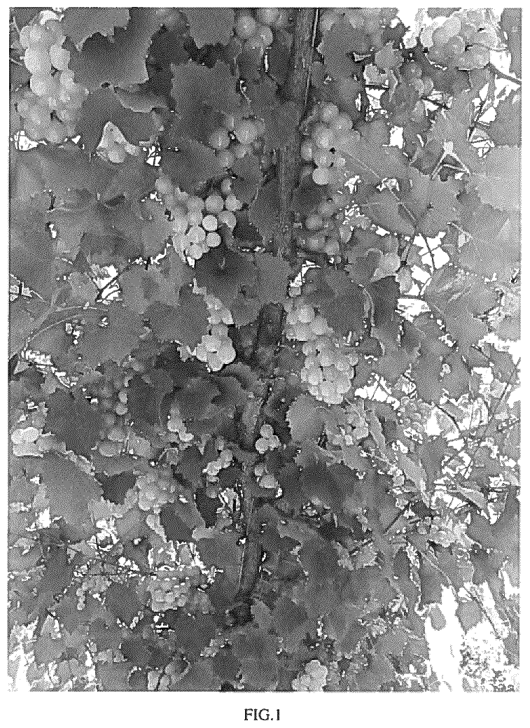

FIG. 2 shows a cluster of ‘JB 12-24-E4-12’ (left) in comparison to the seeded commercial variety ‘Fry’.

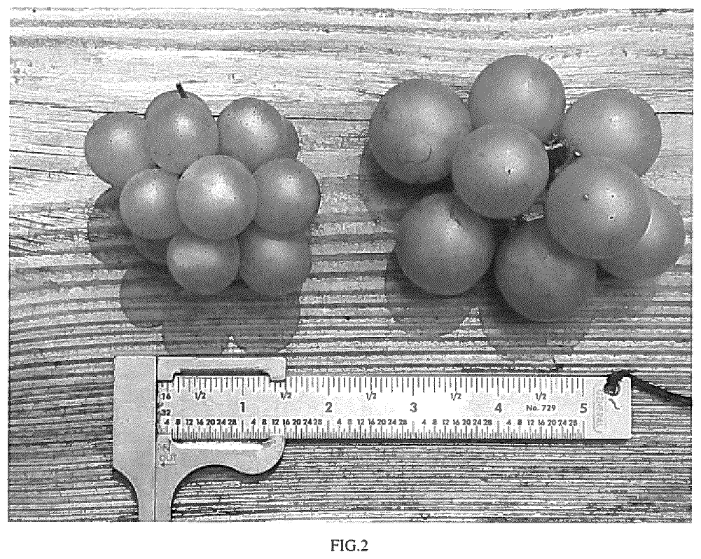

FIG. 3 shows the sectioned fruit of stenospermocarpic seedless ‘JB 12-24-E4-12’ (below) in comparison to that of the seeded commercial variety ‘Fry.’

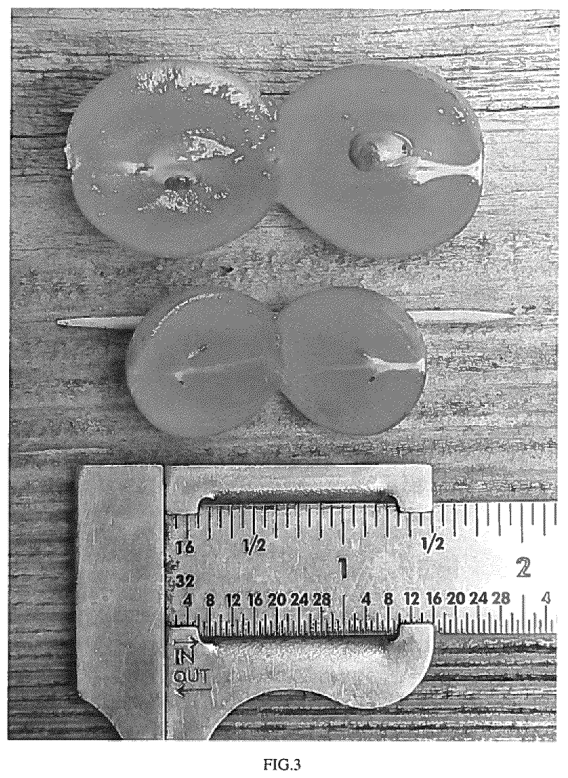

### DESCRIPTION OF THE NEW VARIETY

The following detailed descriptions set for the distinctive characteristics of ‘JB 12-24-E4-12’. Measurements were taken from two own-rooted vines, aged seven and four years old, observed in the field and under artificial daylight halogen illumination in Hillsborough, North Carolina from April to September 2023. Color references are to The R.H.S. Colour Chart of The Royal Horticultural Society of London (R.H.S.) 2007, Fifth Edition.

### Classification:

Family: Vitaceae
Botanical: Hybrid between subgenus Muscadinia Planch. (primarily Vitis rotundifolia) and subgenus Euvitis Planch. (primarily Vitis vinifera, the donor of perfect-floweredness and stenospermocarpic seedlessness)
Designation: ‘JB 12-24-E4-12’

### Plant:

Plant habit and growth: Recumbent, climbing
Size: Large
Height: 6′
Width: 4′
Age at maturity: 3 years
Vigor: High
Productivity: High to the point of overproduction if not properly, severely pruned.
Cold hardiness/winter hardiness/temperature tolerance: One vine, planted in 2016, suffered no apparent winter cold injury after a low temperature of −2° F. in January 2019.

### Trunk:

Diameter: 2.5″ (2016 planting)
Surface texture: Rough
Color: RHS 200C to 200D (old bark), RHS 177B to 177C (freshly exposed)

### Canes:

Diameter:
   Immature: 4.5/32″× 5/32″
   Mature: ⅜″× 5/16″, 3^{rd} proximal internode
Length: Up to 9.5 feet
Surface texture:
   Immature cane: Smooth, barely visible lenticels appear at the 12^{th} internode from shoot tip.
   Mature cane: Rough with numerous raised lenticels
   Form (woody cross-section form): Elliptical
Color:
   Immature: RHS 146A, 12^{th} internode from apex, no anthocyanin
   Mature: RHS 177B
   Internode length (upper mature sun cane): 2″ to 3″
   Time of bud burst: April 8, swollen buds, April 14, green tissue visible, April 15, fully burst

### Tendrils:

Form: Unbranched
Size:
   Length: Up to 5″
   Diameter: 1/32″
Texture and distribution: Smooth, at node opposite petiole, absent at every third node
Color:
   Immature: RHS 144A, 1.5″ in length
   Anthocyanin: No
   Mature: RHS 152A before becoming woody
   Anthocyanin: No

### Growing Tips (Young Shoots):

Pubescence: Present
Color: RHS 144A
   Anthocyanin: Absent
Shape: Shepherd's Crook
Apex: Exposed

### Leaves:

Shape: Orbicular
Apex: Pointed
Base: Sagittate
Margin: Dentate
Length of teeth on margin: ⅛″ to ⅜″
Shape of teeth on margin: Dentate, straight to slightly convex
Texture:
   Immature:
      Upper surface: Smooth
      Lower surface: Slightly rough with raised veins
   Mature:
      Upper surface: Smooth
      Lower surfaces: Slightly rough with raised veins
Size:
   Immature leaf:
      Length: 2.5″
      Width: 2.75″
   Mature leaf:
      Length: 3.25″
      Width: 4.25″
Color:
   Immature leaf:
      Upper surface: RHS 144A
      Lower surface: RHS 144A
   Mature:
      Upper surface: RHS 137A
      Lower surface: RHS 137B
Venation:
   Pattern: Palmate
   Color:
      Upper surface: RHS 146A
      Lower surface: RHS 146B

### Petiolar Sinus: Open, Lyre Shaped

### Petiole:

Length: 2.75″ to 3.25″
Diameter: 1/16″
Color: RHS 152A (Ribs) RHS 144B (Grooves)

### Floral Cluster:

General description and location: Panicle, at node opposite leaf
Quantity of florets per cluster: 79
Length: 1⅝″
Width: 1.5″
Peduncle length: ⅜″-1⅝″

### Inflorescences: Perfect-Flowered

Quantity of inflorescences per shoot: 2-3
Pistil:
   Length: 3/32″
   Diameter: 1.5/32″
   Color: RHS 152B
Filament:
   Length: 2.5/32″
   Color: RHS 193B
Anther:
   Shape: Oblong, Ovate
   Color: RHS 18C
Stamen:
   Form: Upright
   Quantity: 6 to 7/floret
   Length: 3/32″
   Color: Not Applicable
Date of bloom: 6/09 to 06/15
Pollen amount: Abundant
Calyptra: Sheds normally
Calyptra color: RHS 144C

### Fruit:

Time of year of commercial harvest & shipment: First ripe 09/07, Main crop 09/11 to 10/07, small to medium secondary crop on lateral shoots
Cluster:
   General Type: Panicle
   Size: Medium to large for V. rotundifolia, small for V. vinifera
   Length, without peduncle: 2.5″ to 3″
   Width: 2.5″ to 3.5″
   Density: Well-filled
   Shape and weight of cluster: Globular, 1.9 to 3.7 oz.
Peduncle:
   Length: Variable, Range: ⅜″ to 1⅝″. Mean is 0.87″. More vigorous shoots have longer peduncles. Severe pruning to prevent overcropping should result in higher shoot vigor and provide longer peduncles, facilitating cluster harvest. Peduncles shorter than about ⅞″ make cluster harvest more difficult.
   Diameter: 1/16″
   Color: RHS N199B
   Number of berries per cluster: Up to 21
Berry:
   Size: Medium to large
   Shape: Spherical
   Uniformity: Acceptable
   Length: Mean is 13/16″, Max is 15/16″
   Width/Diameter: Mean is 13/16″, Max is 15/16″
   Skin
      Type: Slip Skin
      Skin color: RHS 153D, near ripe; RHS 164B, fully ripe.
      Lenticels: Medium to high in density, size minute up to 0.023″ in diameter, color RHS 165A
      Thickness: 1/16″ as manually peeled from flesh
      Texture: crunchy, relatively tender, fragments easily, non-astringent
      Waxy Bloom: None
   Weight of individual berry: Mean is 4.57 gm on slightly overcropped vines.
      Maximum is 6.5 gm
Pedicel:
   Length: 5/32″
   Diameter: 1/16″
   Color: RHS 199B
   Strength of attachment to berry: Strong, detaches with wet scar, rarely with a broken pedicel attached to the berry, A small percentage will shell in handling and storage.
Flesh:
   Color: RHS 150D, translucent
   Juice color: RHS 155D, clear
   Juice production: high
   Texture: Mucilaginous, almost totally melting
   Brix: Mean when ripe: 18.6°, Individual berries: up to 20.2°.
   Flavor: Fruity, musky
   Fragrance: Flowery, fruity, musky

### Seed Traces:

Length: Minute up to 3/32″
Width: Minute up to 1/32″
Texture: Soft
Perception: Unnoticeable
   Disease and insect resistance:

Both parents of ‘JB 12-24-E4-12’ are resistant to the common muscadine berry rots. ‘JB 12-24-E4-12’ has minimum berry rot incidence under a standard pesticide spray program including fungicides: Manzate Pro-Stick, Nova, and Captan.

Cultural Note: Ripe berries of ‘JB 12-24-E4-12’ appear largely resistant to skin cracking induced by significant rainfall, with only minimal incidence of cracking observed.

### Comparison with Parental Lines and Known Variety

‘JB 12-24-E4-12’ is a distinct variety of grapevine and is distinguished from its parents as described in Table 1 and Table 2:

TABLE 1
Comparison with Female Parental Line

| | | Female parent |
|---|---|---|
| Trait | Variety ‘JB 12-24-E4-12’ | ‘JB99-1-4-15’ |
| Seed | Seedless | Seeded |
| Sex | Perfect-flowered | Female |

TABLE 2
Comparison with Male Parental Line

| | | Male Parent |
|---|---|---|
| Trait | Variety ‘JB 12-24-E4-12’ | ‘JB06-23-1-4’ |
| Fruit size | Larger than male parent | Smaller than |
| | | ‘JB 12-24-E4-12’ |

‘JB 12-24-E4-12’ is most similar to the commercial grapevine variety ‘Tara’ (unpatented). Differences between the varieties are described in Table 3.

TABLE 3
Comparison with Commercial Line

| | | Commercial line |
|---|---|---|
| Characteristic | ‘JB 12-24-E4-12’ | ‘Tara’ |
| Seed | Seedless | Seeded |

I claim:

1. A new and distinct variety of grapevine plant named ‘JB 12-24-E4-12’ as illustrated and described herein.
